(19) United States
(12) Patent Application Publication
(10) Pub. No.: US 2013/0041355 A1
(43) Pub. Date: Feb. 14, 2013

(54) Reducing Damage From A Dielectric Breakdown in Surgical Applications

(71) Applicants: Tammo Heeren, Aliso Viejo, CA (US); Mauricio Jochinsen, Fountain Valley, CA (US)

(72) Inventors: Tammo Heeren, Aliso Viejo, CA (US); Mauricio Jochinsen, Fountain Valley, CA (US)

(21) Appl. No.: 13/207,555

(22) Filed: Aug. 11, 2011

(51) Int. Cl. A61B 18/18

(52) U.S. Cl. 606/5

(57) ABSTRACT

Methods and apparatus are disclosed for reducing damage caused by a dielectric breakdown during surgical application of a pulsed-electric field (PEF) device. By detecting a dielectric breakdown when it occurs or before it occurs, the properties of the pulsed electric field can be adjusted to reduce damage caused by the energy released during the breakdown. A dielectric breakdown or its precursor can be detected optically, acoustically, or electrically.

### TECHNICAL FIELD

The present invention relates generally to the field of eye surgery and more particularly to methods and apparatus for performing eye surgery using high-intensity pulsed electric fields.

### BACKGROUND

Techniques for dissociation and removal of highly hydrated macroscopic volumes of proteinaceous tissue using rapid, variable-direction energy-field flow have been previously disclosed. See Steven W. Kovalcheck in “System for Dissociation and Removal Proteinaceous Tissue,” U.S. patent application Ser. No. 11/608,877, filed on Dec. 11, 2006 and published on Jul. 5, 2007 as U.S. Patent Application Publ. No. 2007/0156129 (hereinafter “the Kovalcheck application”), the entire contents of which are incorporated herein by reference.

As explained in the Kovalcheck application, conventional procedures for vitreoretinal posterior surgery have been based on mechanical or traction methods such as: 1) tissue removal with shear cutting probes (utilizing either a reciprocating or rotary cutter); 2) membrane transaction using scissors, a blade, or vitreous cutters; 3) membrane peeling with forceps and picks; and 4) membrane separation with forceps and viscous fluids. In contrast, the Kovalcheck application introduced a novel tissue removing technique employing a variable-direction, pulsed high-intensity and ultra-short duration disruptive electric field.

In particular, the Kovalcheck application describes a probe for delivering a pulsed, rapid disruptive energy field to soft proteinaceous tissue surrounded by the probe. Once the adhesive mechanism between tissue constituents is compromised by the electric field, fluidic techniques may be used to remove the dissociated tissue. The parameters of the high-intensity electric pulses, such as pulse duration, repetition rate, pulse pattern, pulse train length, and pulse amplitude, can be adjusted to vary the amount of energy delivered and the profile of the energy delivered, to increase the effectiveness of the pulses without over-exposing the vitreous to damaging heat.

### SUMMARY

A dielectric breakdown during the application of a pulsed electric field to tissue may cause damage to the biological organ at the surgical site where the PEF device is inserted. A pulsed-electric field (PEF) surgical device that can prevent or reduce damages caused by a dielectric breakdown is described below.

An example pulsed-electric-field surgical device comprises a pulse generation circuit configured to generate electrical pulses to be applied to a surgical site via electrodes; one or more sensors to detect an attribute characteristic of a dielectric breakdown; a transducer configured to monitor the characteristic to detect that a dielectric breakdown has occurred or is imminent during surgical application of the electrical pulses to the surgical site; and a control circuit configured to adjust a parameter of the PEF device to reduce damage caused by the dielectric breakdown based on the monitored characteristic.

### BRIEF DESCRIPTION OF FIGURES

FIG. 1 illustrates an exemplary probe used in intraocular posterior surgery.

FIG. 2 illustrates an enlarged view of the tip of the probe shown in FIG. 1.

FIG. 3 illustrates an exemplary PEF surgical device.

FIG. 4 illustrates changes of voltage and current during a dielectric breakdown.

FIG. 5 illustrates an exemplary setup for detecting charge balance during a dielectric breakdown.

FIG. 6 illustrates a flow chart showing an exemplary process of reducing damage caused by a dielectric breakdown in a PEF surgical device.

FIG. 7 illustrates series of electric pulses with parameters adjusted during a dielectric breakdown.

### DETAILED DESCRIPTION

During a surgical application involving pulsed electric field (PEF surgery), tissues are vaporized to achieve sufficient flow and low traction. Once vaporization has occurred at a certain site, the dielectric strength in that region reduces dramatically. This reduced dielectric strength may lead to dielectric breakdown. Dielectric breakdown can deposit significant amounts of energy into the volume of tissues, causing shockwaves, heating, and other undesirable effects. Thus, a commercially successful PEF surgical device needs to detect dielectric breakdown or its precursor and adjust the device's operational parameters in order to prevent or limit the undesirable effects of a dielectric breakdown.

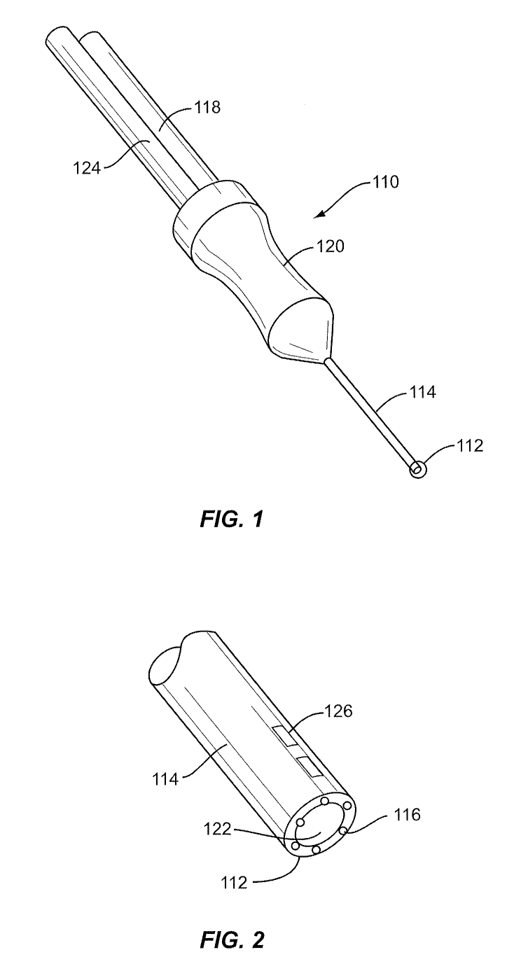

Accordingly, an example PEF device that meets the requirements employs a probe or a needle that can be inserted into an organ, for example, an eye. The probe functions as an electrode for delivery of electric pulses. FIG. 1 illustrates an exemplary PEF probe 110. PEF probe 110 comprises a hollow probe needle 114 extending from handle 120 to probe needle tip 112. PEF probe 110 also comprises an aspiration line 118 and electrical cable/transmission line 124. The details of probe needle 114 and probe needle tip 112 are shown in FIG. 2. At tip 112, a plurality of electrodes 116, connected to electrical cable 124, are exposed. Electrodes 116 surround an aspiration lumen 122, which provides an aspiration pathway to aspiration tube 118. Located on probe needle 114 are also various sensors 126, for example, a photon sensor, a pressure sensor, and/or a thermal sensor, or various meters for measuring voltage or current, etc.

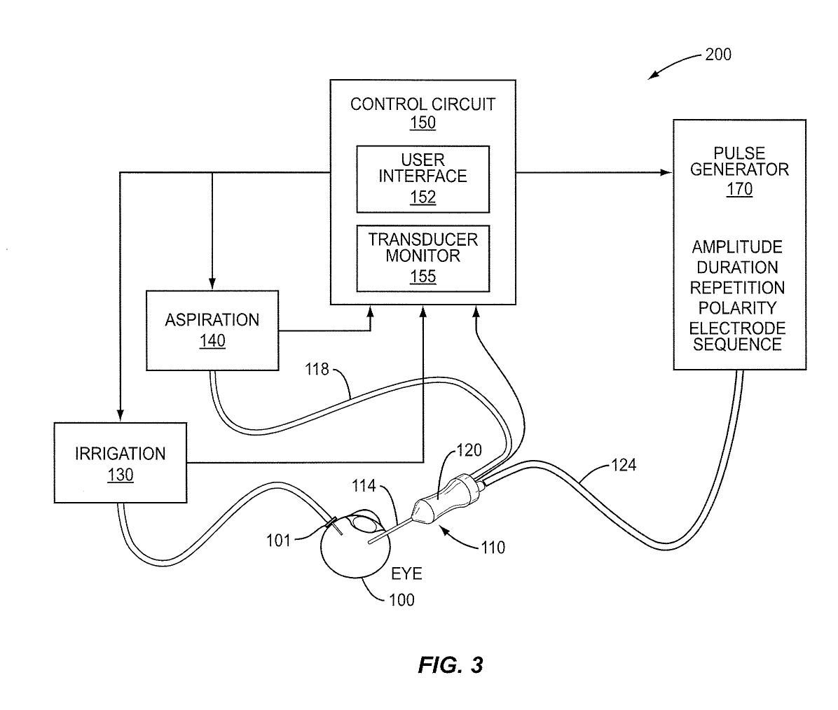

As shown in FIG. 3, the tip 112 of probe 110 may be inserted by a surgeon into the posterior region of an eye 100 via a pars plana approach 101 using handle 120. Using a standard visualization process, vitreous and/or intraocular membranes and tissues are engaged by the tip 112 at the distal end of the hollow probe 114. Irrigation 130 and aspiration 140 mechanisms are activated by control circuit 150, and ultra-short pulsed electric energy, for example, a high-density pulsed electric field, generated by pulse generator 170 is sent to tip 112 via cable 124, creating a disruptive ultra-short-pulsed electrical field within the entrained volume of tissue. The adhesive mechanisms of the tissues at the tip of probe 110 are disassociated by the disruptive pulsed electrical field. The disrupted tissues are then removed with the aid of fluidic techniques. For example, the disassociated tissues are drawn toward probe tip 112 via aspiration through an aspiration line 118 connected to an aspiration lumen 122 in hollow probe needle 114. The tissues enter tip 112 of hollow probe 114 and are removed through aspiration lumen 122 via a saline aspiration carrier to a collection module.

Control circuit 150 controls the operation of PEF device 200. Control circuit 150 includes user interface 152 and transducer monitor 155. User interface 152 allows a user of the device to control the settings and operational parameters of the device before and during the surgery. Transducer monitor 155 monitors one or more relevant surgical parameters at or near the surgical site. The monitored surgical parameters include, but are not limited to, one or more of an irrigation or aspiration flow rate, a sudden flash of light, an intraocular pressure, a temperature, one or more electric properties of the tissue at the surgical site, or the presence of bubble formation.

Transducer monitor 155 is connected to one or more sensors 126 that are located on probe needle 114. Examples of sensors 126 include a flow rate sensor, a photon sensor, a pressure sensor, a thermal sensor, a current sensor, a voltmeter, a bubble formation detector, and so on. In some embodiments, one or more of sensors 126, for example, a current sensor or a voltmeter, may be completely or partially located on probe needle 114. In some embodiments, sensors 126, for example, an aspiration flow sensor, may be located elsewhere. In any case, one or more of such sensors are monitored by transducer monitor 155 of control circuit 150.

In some embodiments, transducer monitor 155 is configured to compare a reading collected by sensors 126 to a predetermined threshold and obtain a comparison result. Based on the comparison result from transducer monitor 155, control circuit 150 in FIG. 3 controls pulse generator 170.

Pulse Generator 170 delivers pulsed DC or gated AC against a low impedance of vitreous and the irrigating solution. The energy storage, pulse shaping, transmission, and load-matching components required by pulse generator 170 are well known to designers of high energy pulse generators and are therefore not detailed further herein. In some embodiments, the peak output voltage of pulse generator 170 is sufficient to deliver up to a 300 kV/cm field strength using the electrodes 116 at the distal end 112 of the hollow surgical probe 114 (see FIG. 2). In some embodiments, peak voltages produced by pulse generator 170 can be of tens of kilovolts.

Pulse generator 170 shown in FIG. 3 delivers electric pulses at an amplitude, a pulse duration, repetition rate, pulse pattern, and pulse train length that are controlled by control circuit 150. Pulse generator 170 is configured to tune pulse duration and repetition rate, and in some embodiments is configured to generate a stepwise continual change in the direction of the electrical field by switching between electrodes, reversing polarity between electrodes or a combination of both in an array of electrodes at the tip 112 of probe needle 114.

Generally, the electric pulses generated by pulse generator 170 are of short duration relative to the dielectric relaxation time of protein complexes. In some embodiments, pulse durations are in the nanosecond range. Optimal operational parameters of the pulse generator 170 can be pre-determined. For example, the pulse duration, repetition rate, and pulse train length (i.e., duty cycle) can be chosen to avoid the development of thermal effects (“cold” process).

Operational parameters of pulse generator 170 can be set before a surgical operation according to different factors, such as patient's conditions, treatment location, treatment type, cumulative or averaged amount of delivered energy, etc. The operational parameters of pulse generator 170 can be adjusted dynamically during a surgical operation as well.

Normally, the rapid changes of direction of the electrical field create disorder in the electric field, without causing dielectric breakdown of the tissues and fluid at the surgical site between the electrodes and without adverse thermal effects. However, during a PEF surgery, the energy from the PEF electric pulses vaporizes a small amount of tissues at the surgical site to facilitate the removal of the extracted tissues by ensuring sufficient flow and achieving low traction. The vaporized tissues reduce the dielectric strength at that surgical site, which can lead to a dielectric breakdown. When a dielectric breakdown occurs, a significant amount of energy may be deposited at the surgical site and may cause undesired effects such as shockwaves or heating. Therefore, it is crucial to detect that a dielectric breakdown has occurred or is imminent, and adjust the pulsed electric fields accordingly to avoid or reduce damage to the vitreous.

A dielectric breakdown is generally accompanied by a flash, a burst of pressure wave, and/or changes in the current or voltage associated with the electric field applied at the surgical site. A dielectric breakdown is caused by a sudden reduction of dielectric strength of the tissue and fluid at the surgical site. The reduced dielectric strength will significantly affect the electric pulses delivered at the surgical site. For example, the voltage across the surgical site may drop significantly due to the reduced dielectric strength.

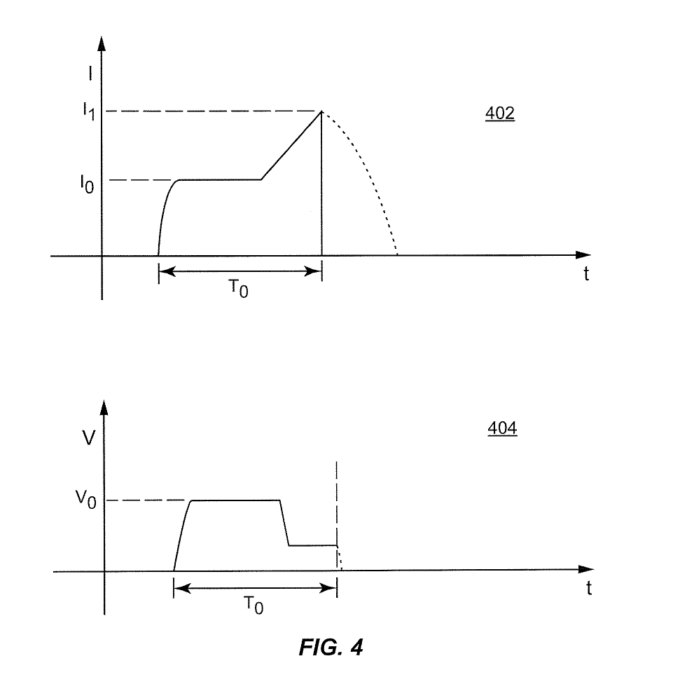

FIG. 4 illustrates the sudden change of electrical properties at the surgical site during a dielectric breakdown. T₀ indicates the pulse duration. In FIG. 4, diagram 402 shows that the current level at the surgical site increases drastically from I₀ to I₁ in the middle of an electric pulse when a dielectric breakdown happens. Diagram 404 shows that the voltage detected at the surgical site drops significantly from V₀ to V₁ during a dielectric breakdown. A sudden increase of current at the surgical site may deposit a large amount of heat or induce flashes or pressure waves similar to a tiny lightning flash, causing damages to the biological organ at the surgical site.

Therefore it is desirable, in order to reduce damage caused by a dielectric breakdown, to detect a dielectric breakdown right after it happens, or to detect its precursor. Several techniques are possible and may be used alone or in combination. For example, a photon sensor incorporated at the tip of PEF probe 110 can be used to detect the flash associated with a dielectric breakdown. A pressure sensor can be used to detect the pressure wave front associated with a breakdown. A voltmeter installed at the tip of probe needle 114 can measure the voltage applied to the tip of probe 114 to detect a sudden voltage drop. A current sensor at the tip of probe 114 can measure the strength of the electric current passing through probe 114 to detect a sudden increase of electric current.

Another indication of an imminent dielectric breakdown is a non-zero charge balance. A linear load fed with a bipolar voltage or current exhibits charge balance, i.e.,

$${\int }_{{t}_{0}}^{t} I d t = 0$$

On the other hand, a dielectric breakdown is a non-symmetric event and therefore results in:

$${\int }_{{t}_{0}}^{t} I d t \neq 0.$$

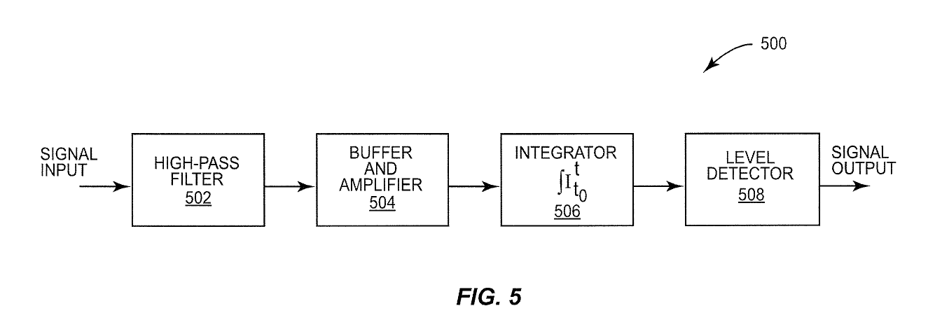

FIG. 5 illustrates an exemplary charge detector 500 for detecting charge built-up. All or parts of charge detector 500 may be installed within or close to PEF probe 110. Charge detector 500 receives an input signal that is equivalent to the current delivered to the surgical site, and employs a High-Pass Filter 502 to filter out noises and retain the desired electric signals. The filtered signals pass through a buffer/amplifier 504 and are fed to an integrator 506 for computation of the net charge built-up. The integration constant is chosen such that both polarities of a bipolar pulse are captures. The result of integrator 506 is input into a level detector 508 to determine whether there is a significant non-zero charge balance, thus indicating an imminent dielectric breakdown. Level detector 508 is configured to compare the result of integrator 506 to a threshold that may be predetermined based on patient's conditions, initial operational parameters of PEF device 200, and other factors.

The output signal from level detector 508 and/or the readings of various sensors/electrical meters 126 are fed to transducer monitor 155 to facilitate detection of a dielectric breakdown. Transducer monitor 155 is configured to compare the data collected by sensors 126 to a threshold to determine whether a dielectric breakdown is imminent or whether a dielectric breakdown has occurred, and, in some cases, the scale of the dielectric breakdown. For example, the threshold may correspond to a predetermined voltage drop, over which a dielectric breakdown will most likely occur. The threshold may correspond to an increase of current, which is predetermined to be a likely precursor of a dielectric breakdown. In the case where charge detector 500 is employed, the detected charge built-up is compared to a charge balance threshold. The charge balance threshold may be set to zero or some other values.

Based on the sensor data and/or the result of the comparison between the sensor data and one or more predetermined thresholds, transducer monitor 155 instructs pulse generator 170 to adjust the properties of the electrical pulses. As noted above, one or more characteristics of the series of electrical pulses applied to the surgical site within the eye may be tuned to the properties of the intraocular tissues, in some embodiments. In some cases, multiple pulse patterns may be employed to address the heterogeneity of intraocular tissue. Characteristics that may be tuned include a pulse amplitude, a pulse shape, a pulse repetition rate, and a pulse train length. Other characteristics applicable to one or more bursts of electrical pulses, any of which might be tuned, include, but are not limited to: a pulse frequency for at least one burst of electrical pulses, a pulse duty cycle for at least one burst of electrical pulses, a burst repetition rate for two or more bursts of electrical pulses, a pulse amplitude for one or more electrical pulses, a pulse duration for one or more electrical pulses, a pulse rise-time for one or more electrical pulses, a pulse fall-time for one or more electrical pulses, and a pulse shape for one or more of the electrical pulses.

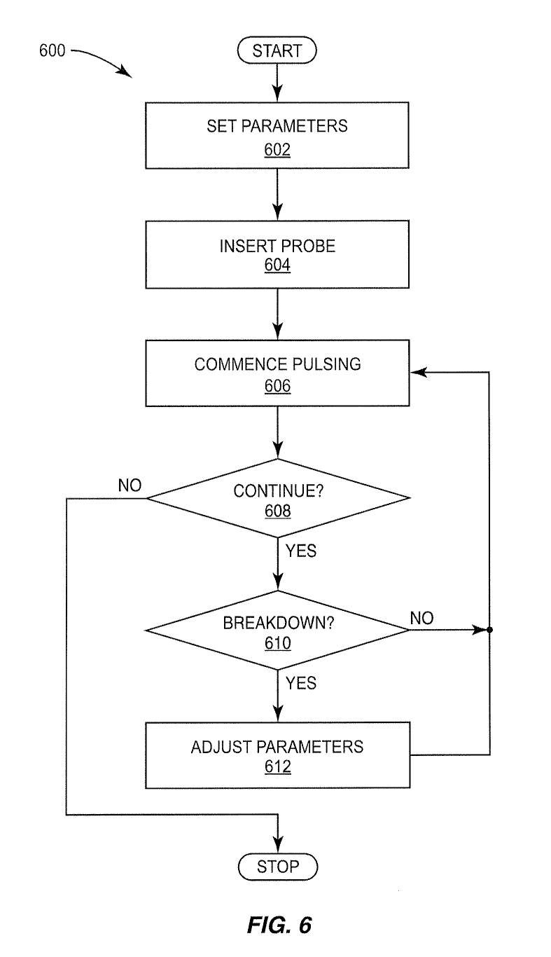

FIG. 6 is a flow chart illustrating a PEF surgical process. At the start of the surgery, initial operational parameters of PEF device 200 are properly set (block 602). During the surgery, PEF probe 110 is first inserted at the surgical site (block 604) and pulsing of the electric fields at the surgical site is then commenced (block 606). Throughout the surgery, surgical parameters are monitored to determine whether to continue applying pulsed electric fields at the surgical site (block 608). If it is decided that the application of pulsed electric fields should not continue, pulsing of the electric fields is stopped. If it is decided that the application of pulsed electric fields should continue, readings of various sensors/meters 126 or charge level detector 508 are fed to control circuit 150 as input data to determine whether a dielectric breakdown is imminent or has occurred (block 610). If it is determined that no dielectric breakdown is imminent or has occurred, pulsing of the electric fields is either resumed or continued. If it is determined that a dielectric breakdown has occurred or is imminent, control circuit 150 may be configured to analyze the input data and determine one or more characteristics of the dielectric breakdown, such as, the scale of the breakdown. Based on the one or more determined characteristics of the dielectric breakdown, control circuit 150 commands pulse generator 170 to adjust its operational parameters in response to the imminent or detected dielectric breakdown, for example, by reducing the strength, duration, and/or shape of the electric pulses delivered to the surgical site (block 612). After the operational parameters of PEF device 200 have been properly adjusted, pulsing of electric fields with newly adjusted attributes is resumed or continued at the surgical site (block 606).

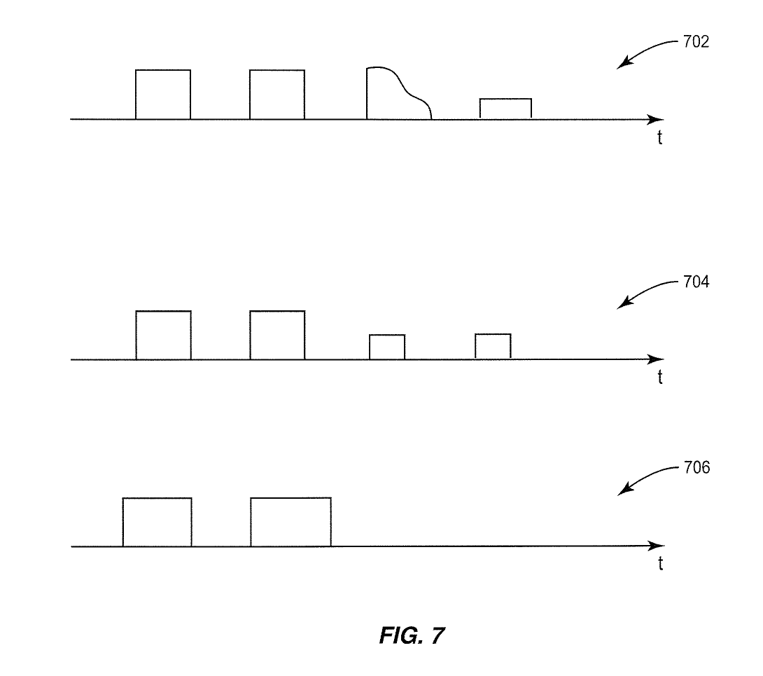

The operational parameters, such as the voltage of the pulses, may be adjusted in the middle of an electric pulse, as shown in diagram 702 in FIG. 7. Alternatively, the operational parameters, such as the duration and voltage of the pulses, may be adjusted in between two electric pulses (diagram 704). The electric pulses may be turned off completely as well (diagram 706). By dynamically adjusting the operational parameters of pulse generator 170 in response to an imminent dielectric breakdown or a dielectric breakdown, PEF device 200 can prevent or reduce damages caused by a dielectric breakdown.

In various applications, the apparatus and techniques described herein may be applied to remove all of the posterior vitreous tissue, or specific detachments of vitreous tissue from the retina or other intraocular tissues or membranes could be realized. Engagement, disruption and removal of vitreous tissue, vitreoretinal membranes, and fibrovascular membranes from the posterior cavity of the eye and surfaces of the retina are critical processes pursued by vitreoretinal specialists, in order to treat sight-threatening conditions such as diabetic retinopathy, retinal detachment, proliferative vitreoretinopathy, traction of modalities, penetrating trauma, epi-macular membranes, and other retinopathologies. Though generally intended for posterior intraocular surgery involving the vitreous and retina, it can be appreciated that the techniques described herein are applicable to anterior ophthalmic treatments as well, including traction reduction (partial vitrectomy); micelle adhesion reduction; trabecular meshwork disruption, manipulation, reorganization, and/or stimulation; trabeculoplasty to treat chronic glaucoma; Schlemm's Canal manipulation, removal of residual lens epithelium, and removal of tissue trailers. Applicability of the disclosed apparatus and methods to other medical treatments will become obvious to one skilled in the art, after a thorough review of the present disclosure and the attached figures.

The preceding descriptions of various methods and apparatus for controlling the application of high-intensity pulsed electric field energy during eye surgery are given for purposes of illustration and example. Those skilled in the art will appreciate that the present invention may be carried out in other ways than those specifically set forth herein without departing from essential characteristics of the invention. The present embodiments are thus to be considered in all respects as illustrative and not restrictive, and all changes coming within the meaning and equivalency range of the appended claims are intended to be embraced therein.

What is claimed is:

1. A method of reducing damage caused by dielectric breakdown during surgical application of a Pulsed-Electric-Field (PEF) device to ocular tissue, comprising:
detecting a characteristic indicating that a dielectric breakdown in the ocular tissue has occurred or is imminent; and
adjusting a parameter of said PEF device based on the detected characteristic.

2. The method of claim 1, wherein the detecting of a characteristic indicating that a dielectric breakdown in the ocular tissue has occurred or is imminent comprises detecting a flash associated with the dielectric breakdown.

3. The method of claim 1, wherein the detecting of a characteristic indicating that a dielectric breakdown in the ocular tissue has occurred or is imminent comprises detecting a pressure wave front associated with the dielectric breakdown.

4. The method of claim 1, wherein the detecting of a characteristic indicating that a dielectric breakdown in the ocular tissue has occurred or is imminent comprises detecting an electrical measurement of said surgical application and comparing the electrical measurement to a threshold.

5. The method of claim 4, wherein said electrical measurement is a measurement of a drop of voltage and the threshold corresponds to a voltage drop predetermined to cause the dielectric breakdown.

6. The method of claim 4, wherein said electrical measurement is a measurement of an increase of current and the threshold corresponds to an increase of current predetermined to cause the dielectric breakdown.

7. The method of claim 4, wherein said electrical measurement is a measurement of a charge balance and the threshold corresponds to a threshold charge balance.

8. The method of claim 4, wherein said electrical measurement is a measurement of a delivered energy.

9. The method of claim 1, wherein the adjusting of a parameter of said PEF surgical device comprises adjusting the pulse duration of the electrical field of the PEF surgical device.

10. The method of claim 1, wherein the adjusting of a parameter of said PEF device comprises adjusting the pulse voltage of the electrical field of the PEF surgical device.

11. The method of claim 1, wherein the adjusting of a parameter of said PEF device comprises adjusting the pulse repetition rate of the electrical field of the PEF surgical device.

12. The method of claim 1, wherein said surgical application is vitrectomy.

13. A Pulsed Electric Field (PEF) device, comprising:
a pulse generation circuit configured to generate electrical pulses to be applied to ocular tissue via electrodes;
one or more sensors configured to measure an attribute characteristic of a dielectric breakdown in the ocular tissue;
a transducer configured to monitor the measured attribute to detect that a dielectric breakdown has occurred or is imminent; and
a control circuit configured to adjust a parameter of the PEF device based on the measured attribute to reduce damage caused by the dielectric breakdown.

14. The PEF device of claim 13, wherein the parameter of the PEF device being adjusted to reduce damage caused by the dielectric breakdown comprises a pulse duration for each of the electrical pulses.

15. The PEF device of claim 13, wherein the parameter of the surgical device being adjusted to reduce damage caused by a dielectric breakdown comprises a pulse voltage of the electrical pulses.

16. The PEF device of claim 13, wherein the one or more sensors are configured to detect a flash associated with the dielectric breakdown in the ocular tissue.

17. The PEF device of claim 13, wherein the one or more sensors are configured to take an electrical measurement in the ocular tissue.

18. The PEF device of claim 17, wherein the electrical measurement comprises a drop of voltage in the ocular tissue.

19. The PEF surgical device of claim 17, wherein the electrical measurement comprises a charge balance in the ocular tissue.

20. The PEF surgical device of claim 17, wherein the electrical measurement comprises an increase of current in the ocular tissue.

21. The PEF surgical device of claim 17, wherein the transducer is further configured to compare the electrical measurement to a predetermined threshold and the control circuit is further configured to adjust the parameter of the PEF device based on the comparison.
